(19) United States
(12) Patent Application Publication
(10) Pub. No.: US 2017/0138889 A1
(43) Pub. Date: May 18, 2017

(54) BIOLOGICAL TEST SHEET

(71) Applicant: APEX BIOTECHNOLOGY CORP., Hsinchu (TW)

(72) Inventors: Sz-Hau Chen, Taipei City (TW); Ya-Chun Chang, Hsinchu County (TW); Chia-Wen Tsai, Hsinchu City (TW); Mon-Wen Yang, Hsinchu (TW)

(73) Assignee: APEX BIOTECHNOLOGY CORP., Hsinchu (TW)

(21) Appl. No.: 15/345,484

(22) Filed: Nov. 7, 2016

Related U.S. Application Data
(60) Provisional application No. 62/254,728, filed on Nov. 13, 2015.

(30) Foreign Application Priority Data
Oct. 11, 2016 (TW) 105132775

(51) Int. Cl. G01N 27/327; C12Q 1/00

(52) U.S. Cl. CPC G01N 27/3277; G01N 27/3272; C12Q 1/001

(57) ABSTRACT

A biological test sheet includes an insulating substrate, an electrode structure, a first insulating septum and an insulating layer. The electrode structure is disposed on the insulating substrate and has at least one top surface and at least one side surface, and the side surface is connected between at least one fringe of the top surface and the insulating substrate. The first insulating septum is disposed on the insulating substrate and partially covers the electrode structure. The first insulating septum has a notch, and the notch exposes a first segment of the electrode structure. The insulating layer covers the fringe of the top surface and the side surface at the first segment of the electrode structure.

### CROSS-REFERENCE TO RELATED APPLICATION

This application claims the priority benefits of U.S. provisional application No. 62/254,728, filed on Nov. 13, 2015, and Taiwan application no. 105132775, filed on Oct. 11, 2016. The entirety of each of the above-mentioned patent applications is hereby incorporated by reference herein and made a part of this specification.

### BACKGROUND OF THE INVENTION

1. Field of the Invention

The invention generally relates to a test sheet, and more particularly, to a biological test sheet.

2. Description of Related Art

In modern days, with an abundance of food, more and more diseases are caused by eating habits. According to statistics of World Health Organization (WHO), in year 2000, about 150 million people worldwide suffer from cardiovascular diseases. For people in need of long-monitoring of body conditions (e.g., blood sugar, blood lipid and so forth), a reliable electrochemical measurement system has thus become increasingly important and an indispensable tool in daily life.

A traditional biological test sheet (e.g., an electrochemical test sheet) is consisted of an insulating substrate, an electrode structure disposed on the insulating substrate and an insulating septum covering a portion of the electrode structure. During usage, a user needs to insert a sample (e.g., blood, body fluid and so forth) to a portion of the electrode structure exposed by the insulating septum so that the sample can react with a reaction layer to generate an oxidation-reduction signal to the electrode structure for performing a test. During the process of fabricating the electrode structure, side surfaces of the electrode structure are prone to burr production and fringes of a top surface of the electrode structure are prone to have bevels, thereby causing a surface area of the electrode structure to produce an unexpected change. In addition, in order to increase a bonding force between the electrode layers of the electrode structure, a bonding layer made of copper powders, metallic salts or so forth is typically disposed between the electrode layers; however, portions of the bonding layer at the side surfaces of the electrode structure are susceptible to produce a stripping phenomenon, which can also cause the surface area of the electrode structure to produce an unexpected change. Moreover, a copper electrode is generally used for the electrode layer at the bottom of the electrode structure so as to easily bond with the insulating substrate; however, the copper electrode surface is prone to be oxidized to produce a shielding phenomenon, which can also cause the surface area of the electrode structure to produce an unexpected change. The above factors can all cause a contract area between the electrode structure and a reactant to result in an unexpected value, thereby influencing a normal reception of an oxidation-reduction signal and lowering the accuracy of a test result.

### SUMMARY OF THE INVENTION

The invention provides a biological test sheet capable of enhancing a test accuracy.

A biological test sheet of the invention includes an insulating substrate, an electrode structure, a first insulating septum and an insulating layer. The electrode structure is disposed on the insulating substrate and has at least one top surface and at least one side surface, and the side surface is connected between at least one fringe of the top surface and the insulating substrate. The first insulating septum is disposed on the insulating substrate and partially covers the electrode structure. The first insulating septum has a notch, and the notch exposes a first segment of the electrode structure. The insulating layer covers the fringe of the top surface and the side surface at the first segment of the electrode structure.

In one embodiment of the invention, a junction between the fringe of the top surface and the side surface is covered by the insulating layer.

In one embodiment of the invention, the insulating layer contacts the fringe of the top surface and the side surface.

In one embodiment of the invention, a second segment of the electrode structure is covered by the first insulating septum, and the insulating layer covers the fringe of the top surface and the side surface at the second segment of the electrode structure.

In one embodiment of the invention, the insulating layer covers a portion of the insulating substrate exposed by the notch.

In one embodiment of the invention, the insulating layer covers a portion of the insulating substrate exposed by the notch, a portion of the insulating substrate outside the notch and a portion of the electrode structure outside the notch.

In one embodiment of the invention, the biological test sheet further includes a reaction layer, wherein the reaction layer is disposed in the notch.

In one embodiment of the invention, the biological test sheet further includes a second insulating septum, wherein the second insulating septum is disposed on the first insulating septum and covers the notch.

In one embodiment of the invention, a surface of the second insulating septum faces towards the insulating substrate, and a material of the surface at the notch includes a hydrophilic material.

In one embodiment of the invention, the second insulating septum has a ventilation hole, and the ventilation hole is aligned with the notch.

In view of the above, in the biological test sheet of the invention, the insulating layer covers the fringe of the top surface of the electrode structure and covers the side surface of the electrode structure. That is, the electrode structure contacts a reactant merely with a portion of the top surface that is not covered by the insulating layer. As such, even if the side surface of the electrode structure has burr, the fringe of the top surface of the electrode structure has a bevel, the side surface of the electrode structure produces a stripping phenomenon or a shielding phenomenon, and thereby cause surface areas of the top surface and the side surface of the electrode structure to produce unexpected changes, a contact area between the electrode structure and the reactant would not result in an expected value, and thus the accuracy of a test result can be effectively increased.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a perspective view illustrating a biological test sheet according to an embodiment of the invention.

FIG. 2 is an exploded view of the biological test sheet of FIG. 1.

FIG. 3 is a top view of the biological test sheet of FIG. 1.

FIG. 4 is a cross-sectional view illustrating an insulating substrate and an electrode structure of FIG. 2 along a line I-I.

FIG. 5A illustrates current and voltage relations, obtained during multiple tests, of a biological test sheet not configured with an insulating layer of FIG. 4 therein.

FIG. 5B illustrates current and voltage relations, obtained during multiple tests, of a biological test sheet configured with the insulating layer of FIG. 4 therein.

FIG. 6 is a schematic view illustrating the biological test sheet of FIG. 1 being connected to a biological measuring instrument.

FIG. 7 is a top view of a biological test sheet according to another invention of the application.

FIG. 8 is a top view of a biological test sheet according to another invention of the application.

### DESCRIPTION OF THE EMBODIMENTS

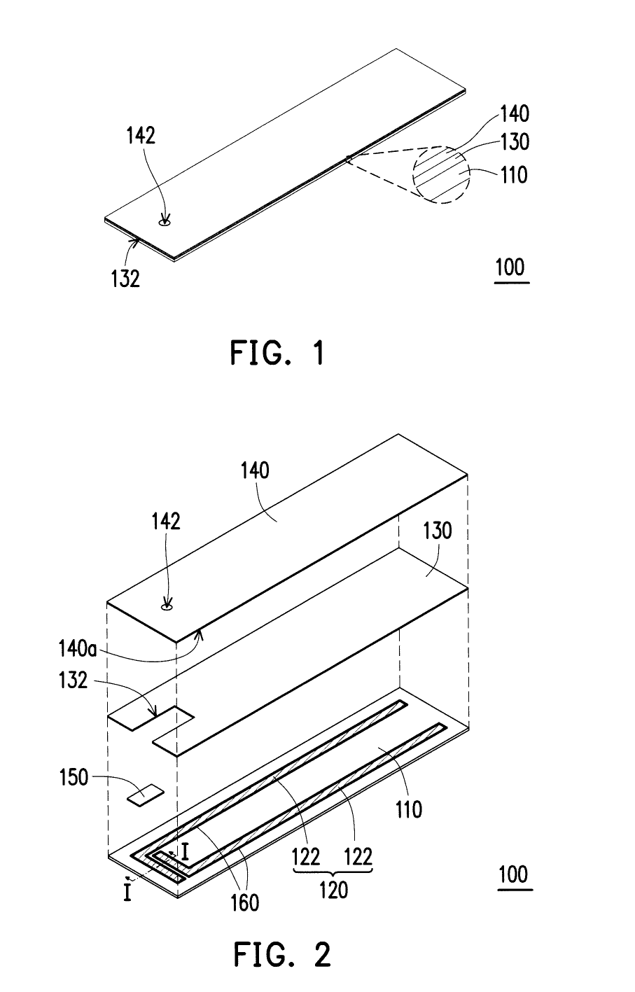

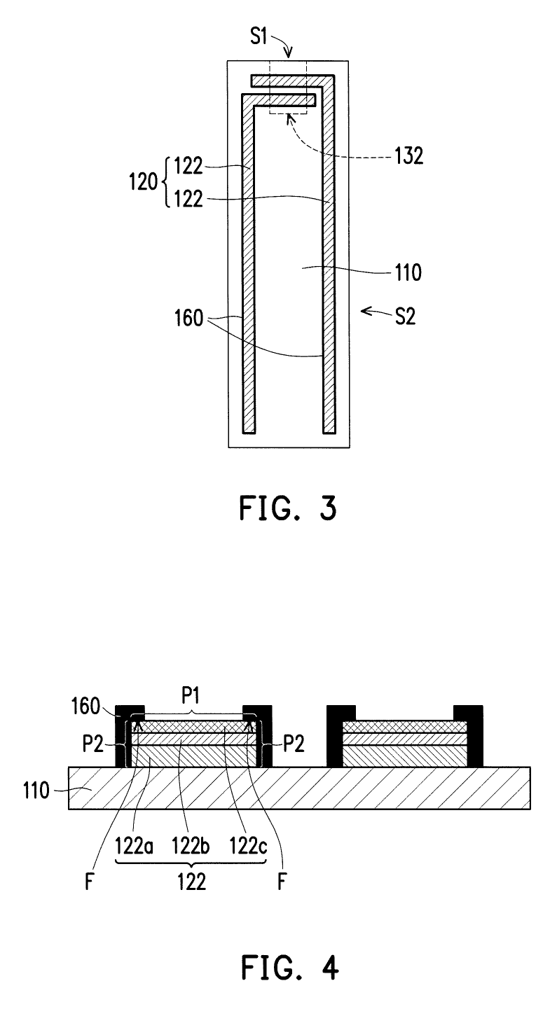

FIG. 1 is a perspective view illustrating a biological test sheet according to an embodiment of the invention. FIG. 2 is an exploded view of the biological test sheet of FIG. 1. FIG. 3 is a top view of the biological test sheet of FIG. 1. For the clarity of the drawings, a first insulating septum 130 and a second insulating septum 140 from FIG. 1 and FIG. 2 are not illustrated in FIG. 3, and FIG. 3 shows a location of a notch 132 of the first insulating septum 130 with dashed-line. Referring to FIG. 1 and FIG. 2, a biological test sheet 100 of the present embodiment includes an insulating substrate 110, an electrode structure 120, a first insulating septum 130, a second insulating septum 140 and a reaction layer 150. In the present embodiment, the biological test sheet 100 is configured to receive an electrochemical test sheet of a user sample so as to measure values of blood sugar, cholesterol, uric acid, lactic acid, hemoglobin and so forth in human body. However, types and measurement items of the biological test sheet 100 are not limited thereto.

In the present embodiment, the insulating substrate 110 is a substrate having a flat and smooth surface, and having electrical insulation and thermal resistance of 40° C. to 120° C. A material of the insulating substrate 110 may include polyvinyl chloride (PVC), glass fiber (FR-4), polyester suphone, a bakelite board, polyethylene terephthalate (PET), polycarbonate (PC), polypropylene (PP), polyethylene (PE), polystyrene (PS), glass plate, ceramic or any combination of the above materials, but the invention is not limited thereto.

The electrode structure 120, for example, includes two electrodes 122 and are disposed on the insulating substrate 110, and the first insulating septum 130 is disposed on the insulating substrate 110 and partially covers the electrode structure 120. Specifically, the first insulating septum 130 has a notch 132, the notch 132 exposes a first segment Si of the electrode structure 120 (as marked in FIG. 3, such that the electrode structure 120 is located in a segment within the range of the notch 132), and the first insulating septum 130 covers a second segment S2 of the electrode structure (as marked in FIG. 3, such that the electrode structure 120 is located at a segment outside the range of the notch 132). The reaction layer 150 is disposed in the notch 132 of the first insulating septum 130 by means of, for example, dispensing or printing, and the second insulating septum 140 is disposed on the first insulating septum 130 and at least covers a portion of the notch 132. The user can insert the sample to a position of the first segment S1 of the electrode structure 120 via an end of the notch 132 as shown in FIG. 1, so as to enable the sample to react with the reaction layer 150 at the position and to generate an oxidation-reduction signal to the electrode structure 120 for performing a test.

In the present embodiment, the electrode structure 120 is, for example, fabricated by means of sputtering, vapor deposition, electroplating, ultrasonic spray, pressurized spray, photolithography, stripping lithography, laser ablation or other suitable method. FIG. 4 is a cross-sectional view illustrating the insulating substrate and the electrode structure of FIG. 2 along a line I-I. Referring to FIG. 4, in detail, each of the electrodes 122 of the present embodiment includes electrode layers 122a, 122b and 122c, and material of the electrode layers 122a, 122b and 122c are respectively copper, zinc and gold, but not limited thereto. In other embodiment, the materials of each of the electrodes 122 may include appropriate conductive or semiconductor materials, such as palladium, gold, platinum, silver, iridium, carbon, indium tin oxide, indium tin oxide, copper, aluminum, gallium, iron, amalgam, tantalum, titanium, zirconium, nickel, osmium, rhenium, rhodium, palladium, organic metal, conductive carbon powder and so forth, or at least one metal combination of other known conductive materials or semiconductor materials. In order to increase bonding forces between the electrode layers 122a, 122b and 122c, a bonding layer made of copper powders, metallic salts or so forth can be disposed between the electrode layers 122a and 122b and between the electrode layers 122b and 122c. In addition, materials of the first insulating septum 130 and the second insulating septum 140 are, for example but no limited to, polyvinyl chloride (PVC) insulation tape, ethylene terephthalate insulating tape, thermal drying insulating varnish or UV-curable insulating varnish.

In the present embodiment, the reaction layer 150 may include at least one active substance and a conductive medium for producing a chemical reaction with the sample. The active substance may include immobilized or non-immobilized enzymes, such as glucose oxidase, antigen, antibody, microbial cells, animal and plant cells, compositions of animal and plant tissues with biological recognition ability. The conductive medium can be used to receive an electron generated after the active substance reacts with the sample, and transfer the electron to a biological measuring instrument through an electrode unit. Compositions thereof, for example include but not limited to, enzymes (e.g., glucoamylase), conductive medium (e.g., potassium ferricyanide), phosphate buffer, protective agent (e.g., protein, dextrin, glucan, amino acid and so forth).

Referring from FIG. 2 to FIG. 4, the biological test sheet 100 of the present embodiment includes an insulating layer 160 configured to cover a partial surface of the electrode structure 120. In detail, each of the electrodes 122 of the electrode structure 120 has a top surface P1 and two opposite side surfaces P2, and the two side surfaces P2 are connected between two fringes F of the top surface P1 and the insulating substrate 110. The insulating layer 160 covers the fringes F of the top surface P1 and the side surfaces P2 at the first segment S1 of the electrode structure 120. That is, the electrode structure 120 contacts a reactant (i.e., the aforementioned sample and the reaction layer 150) with a portion of the top surface P1 that does not include the fringes F and is not covered by the insulating layer 160, wherein the insulating layer 160, for example, directly contacts the fringes F of the top surface P1 and the side surfaces P2.

As such, even if the side surfaces P2 of the electrode structure 120 have burrs, the fringes F of the top surface P1 of the electrode structure 120 have bevels, the side surfaces P2 of the electrode structure 120 can easily produce a stripping phenomenon due to the disposition of the bonding layers, the side surfaces P2 of the electrode structure 120 can easily be oxidized to produce a shielding phenomenon due to the material of the electrode layer 122a being copper, and thereby cause the top surface P1 and the side surfaces P2 of the electrode structure 120 to produce unexpected changes, a contact area between the electrode structure 120 and the reactant would not result in an expected value, and thus the accuracy of a test result can be effectively increased. In addition, as shown in FIG. 4, junctions between the fringes F of the top surface P1 and the side surfaces P2 are covered by the insulating layer 160, and thus the reactant can be prevented from infiltrating between the side surfaces P2 and the electrode structure 120 from the junctions.

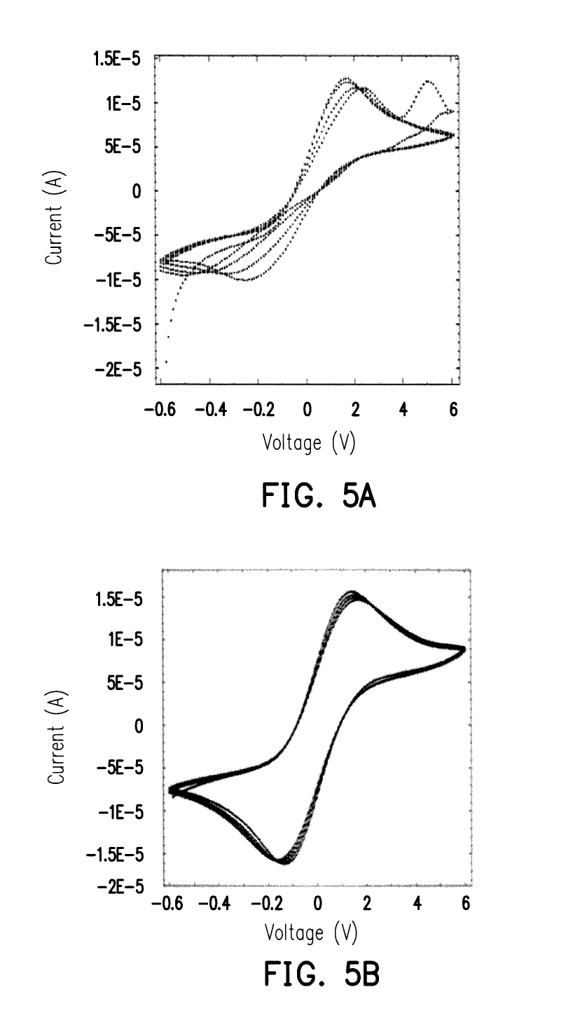

FIG. 5A illustrates multiple cyclic voltammetry tests being performed by a biological test sheet not configured with the insulating layer of FIG. 4 therein. FIG. 5B illustrates multiple cyclic voltammetry tests being performed by a biological test sheet configured with the insulating layer of FIG. 4 therein. As commonly known to those skilled in the art, a cyclic voltammetry test is performed by applying a potential which goes from a start potential to an end potential with a fixed rate and then changes the potential back to the start potential with the same rate, and a redox signal diagram can be generated with this potential test. By comparing between FIG. 5A and FIG. 5B, it can be known that, the multiple cyclic voltammetry tests of the biological test sheet not configured with the insulating layer 160 therein, as shown in FIG. 5A, have a larger variability, which indicates that an oxidation-reduction signal thereof is more susceptible to interference; while the multiple cyclic voltammetry tests of biological test sheet configured with the insulating layer 160 therein, as shown in FIG. 5B, have a smaller variability, which indicates that an oxidation-reduction signal thereof is less susceptible to interference, and thus can increase the accuracy of the test result, as described in the above.

Referring to FIG. 2, a lower surface 140a of the second insulating septum 140 of the present embodiment faces towards the insulating substrate 110, and a material of the lower surface 140a at the notch 132 includes a hydrophilic material. As such, with the hydrophilic characteristics of the hydrophilic material, the sample can be restricted at the notch 132 so as to prevent the accuracy of the test from being influenced due to an unexpected infiltration of the sample. The hydrophilic material can at least be coated on the lower surface 140a of the second insulating septum 140 at a position corresponding to the notch 132, but the invention is not limited thereto.

In the present embodiment, the sample being inserted to the notch 132, for example, is transferred between the insulating substrate 110, the first insulating septum 120 and the second insulating septum 140 through capillary action, so as to be evenly distributed over a coverage of the notch 132. Accordingly, the second insulating septum 140 of the present embodiment has a ventilation hole 142, and the ventilation hole 142 is aligned with the notch 132 to modulate a pressure within the notch 132 so as to facilitate a progress of the capillary action.

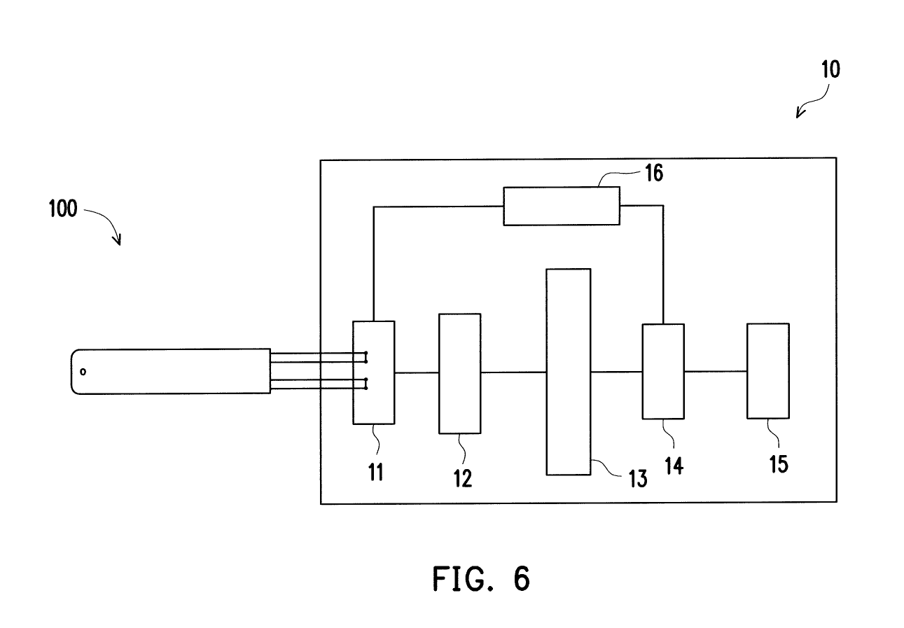

Examples are provided below for describing an operation method of the biological test sheet 100 of the present embodiment. FIG. 6 is a schematic view illustrating the biological test sheet of FIG. 1 being connected to a biological measuring instrument. Referring to FIG. 2 and FIG. 6, the electrode structure 120 as shown in FIG. 2 can be connected to a biological measuring instrument 10 as shown in FIG. 6 so as to form an electrical circuit with the biological measuring instrument 10. The biological measuring instrument 10 includes a connector 11 for external connections, a computational unit 12 for converting concentrations, an analog-to-digital converter (analog to digital converter, ADC) 13, a processor 14, a display 15 and a power supply unit 16. When the power supply unit 16 of the biological measuring instrument 10 applies an electrical signal to the electrode structure 120, the sample and the reaction layer 150 which carry out a reaction at the notch 132 generate a corresponding oxidation-reduction signal for being transmitted to the computational unit 12 of the biological measuring instrument 10 through the connector 11. Afterwards, the reaction signal is converted by the computational unit 12 and outputted to the analog-to-digital converter 13, so as to obtain a digitized reaction signal, wherein the digitized reaction signal is further processed by the processor 14 and/or displayed by the display 15 to show the test result. In other embodiments, the biological test sheet 100 can be operated via other appropriate method, and the invention is not limited to the above.

In the present embodiment, the insulating layer 160, in addition to covering the fringes F of the top surface P and the side surfaces P2 at the first segment S1 (as marked in FIG. 3) of the electrode structure 120, as shown in FIG. 4, further covers the fringes F of the top surface P1 and the side surfaces P2 at the second segment S2 (as marked in FIG. 3) of the electrode structure 120. That is, the fringes F of the top surface P1 and the side surfaces P2 of each of the electrodes 122 of the electrode structure 120 are completely covered by the insulating layer 160. However, the invention does not intend to limit a distribution range of the insulating layer 160, and further examples, accompanied by drawings, are described in detail below.

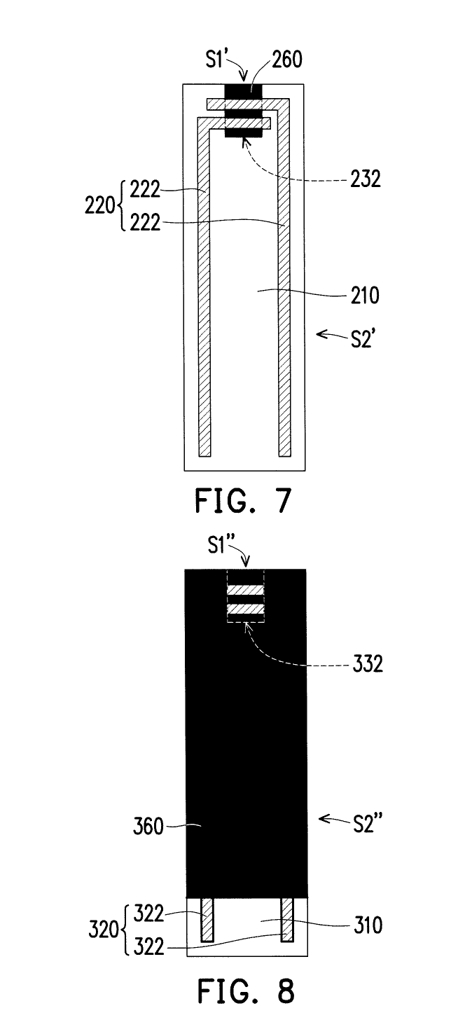

FIG. 7 is a top view of a biological test sheet according to another invention of the application. Configurations and actuations of an insulating substrate 210, an electrode structure 220, electrodes 222, a notch 232, a first segment S1′, and a second segment S2′ of FIG. 7 are similar to that of the insulating substrate 110, the electrode structure 120, the electrodes 122, the notch 132, the first segment S1, and the second segment S2 of FIG. 3, and thus will not be repeated. Differences between the embodiment shown in FIG. 7 and the embodiment shown in FIG. 3 lie in that, the insulating layer 260 covers the fringes of the top surface and the side surfaces of the electrode structure 220 only at the first segment S1′, and the insulating layer 260 further covers a portion of the insulating substrate 210 exposed by the notch 232.

FIG. 8 is a top view of a biological test sheet according to another invention of the application. Configurations and actuations of an insulating substrate 310, an electrode structure 320, electrodes 322, a notch 332, a first segment S1″, and a second segment S2″ of FIG. 8 are similar to that of the insulating substrate 110, the electrode structure 120, the electrodes 122, the notch 132, the first segment S1, and the second segment S2 of FIG. 3, and thus will not be repeated. Differences between the embodiment shown in FIG. 8 and the embodiment shown in FIG. 3 lie in that, the insulating layer 360 further covers a portion of the insulating substrate 310 exposed by the notch 332, a portion of the insulating substrate 310 outside of the notch 332 and a portion of the electrode structure 320 outside of the notch 332. That is, the insulating layer 360 merely exposes a partial portion of the notch 332 that is used to contact with the sample and the reaction layer and a partial portion of the electrode structure 320 (as marked by 322) that is used to connect with the measuring instrument (i.e., the biological measuring instrument 10 shown in FIG. 6).

In summary, in the biological test sheet of the invention, the insulating layer covers the fringes of the top surface of the electrode structure and covers the side surfaces of the electrode structure. That is, the electrode structure contacts the reactant merely with a portion of the top surface that is not covered by the insulating layer. As such, even if the side surfaces of the electrode structure have burrs, the fringes of the top surface of the electrode structure have bevels, the side surfaces of the electrode structure produce a stripping phenomenon or a shielding phenomenon, and thereby cause the surface areas of the top surface and the side surface of the electrode structure to produce unexpected changes, the contact area between the electrode structure and the reactant would not result in an expected value, and thus the accuracy of the test result can be effectively increased.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A biological test sheet, comprising:
an insulating substrate;
an electrode structure, disposed on the insulating substrate and having at least one top surface and at least one side surface, wherein the side surface is connected between at least one fringe of the top surface and the insulating substrate;
a first insulating septum, disposed on the insulating substrate and partially covering the electrode structure, wherein the first insulating septum has a notch, and the notch exposes a first segment of the electrode structure; and
an insulating layer, covers the fringe of the top surface and the side surface at the first segment of the electrode structure.

2. The biological test sheet as recited in claim 1, wherein a junction between the fringe of the top surface and the side surface is covered by the insulating layer.

3. The biological test sheet as recited in claim 1, wherein the insulating layer contacts the fringe of the top surface and the side surface.

4. The biological test sheet as recited in claim 1, wherein a second segment of the electrode structure is covered by the first insulating septum, and the insulating layer covers the fringe of the top surface and the side surface at the second segment of the electrode structure.

5. The biological test sheet as recited in claim 1, wherein the insulating layer covers a portion of the insulating substrate exposed by the notch.

6. The biological test sheet as recited in claim 1, wherein the insulating layer covers a portion of the insulating substrate exposed by the notch, a portion of the insulating substrate outside the notch and a portion of the electrode structure outside the notch.

7. The biological test sheet as recited in claim 1, further comprising a reaction layer, wherein the reaction layer is disposed in the notch.

8. The biological test sheet as recited in claim 1, further comprising a second insulating septum, wherein the second insulating septum is disposed on the first insulating septum and covers the notch.

9. The biological test sheet as recited in claim 8, wherein a surface of the second insulating septum faces towards the insulating substrate, and a material of the surface at the notch comprises a hydrophilic material.

10. The biological test sheet as recited in claim 8, wherein the second insulating septum has a ventilation hole, and the ventilation hole is aligned with the notch.
